(19) United States
(12) Patent Application Publication
(10) Pub. No.: US 2011/0155143 A1
(43) Pub. Date: Jun. 30, 2011

(54) SNORING AND OBSTRUCTIVE SLEEP APNEA PREVENTION AND TREATMENT DEVICE

(76) Inventor: Totada R. Shantha, (US)

(21) Appl. No.: 13/046,080

(22) Filed: Mar. 11, 2011

(51) Int. Cl. A61F 5/56

(52) U.S. Cl. 128/848

(57) ABSTRACT

A snoring and obstructive sleep apnea prevention and treatment device has an oral device portion with a centrally disposed plate portion. The plate portion has a suction cup on its tongue facing side which helps hold it in place and to keep the tongue from moving back towards the throat, thus preventing snoring and sleep apnea. A hood portion fits around the tip of the tongue further preventing the tongue from sliding back into a user's throat. In one embodiment, an inflatable balloon portion is provided to prevent soft tissue from contacting portions of the tongue. In another embodiment, a plate is used in place of the balloons. Additionally, oxygen may be supplied to a posterior portion of the device. Dental adhesives such as denture adhesive may be used to further restrict movement.

### BACKGROUND OF THE INVENTION

Snoring, hypopnea and obstructive sleep apnea (OSA) are caused by the vibrating soft palate; soft tissue of the nasal and oral pharynx, relaxed tongue moving backwards towards the oral pharynx blocking of the air passageway through the pharynx, or lingual compartment during sleep obstructing air passage through the naso and oropharynx. Other causes include; the loose tissue within the mouth cavity including the flaccid tongue, the pharyngeal folding, tonsillar pillars, and the muscular uvula with the soft palate-called the pharyngeal arch which has a propensity to vibrate as tidal air flows past narrow air passages while asleep causing snoring and obstructive sleep apnea.

Snoring is an inspiratory sound which arises in the course of person's sleep and due to the narrowing of the naso and oropharyngeal airway. The sounds of snoring are generated by vibration of soft tissues of oropharynx such as the soft palate, uvula, tongue, lips, the posterior faucial pillars of the tonsils, pharyngeal folds, posterior, and lateral pharyngeal wall and epiglottis in the upper airway; soft palate and uvula are the main culprits (FIGS. 1, 2, 3).

Many causes for the narrowing of the nasal pharyngeal airway, especially during sleep exist besides the flaccid soft palate and the tongue. People who snore rarely make snoring sounds when breathing while awake in the same position that is associated with snoring when asleep (FIG. 1). The reason being that the wide awake conscious person has watchful control of various muscles of the upper airway so as to prevent the vibrations that cause snoring to occur (FIG. 1). During sleep, the motor neurons that control skeletal muscles are inhibited from sending instructions to make them active and increase the tone of these muscles. This physiological process in sleep results in flaccid muscles that permit soft tissue to sag and collapse into the pharyngeal airway and as a result snoring and OSA strike (FIGS. 2 and 3).

It has been estimated that up to 45% of all adults snore sporadically with about 25% being constant snorers. It is known that the snoring increases with advancing age and it has been observed that about 50% of men and 40% of women are habitual snorers by the age of 60. (Lugaresi et al, “Snoring: Pathogenic, Clinical and Therapeutic Aspects”, Reported in Principles and Practice of Sleep Medicine (Kryger et al, Editors 1989) at pp. 494-500).

One needs to discriminate the difference between non obstructive snorers (FIG. 2) from the obstructive sleep apnea snorers ((FIG. 3) and hypopnea (FIG. 2). Hypopnea is a medical term that involves episodes of shallow breathing or an abnormally low respiratory rate and it may not be due to naso-oropharyngeal airways. This differs from sleep apnea in that there remains some flow of air. Hypopnea events may happen while asleep or while awake. It's abnormally shallow breathing lasting at least ten seconds. In the context of diagnosis and treatment of sleep disorders, a hypopnea event is not considered to be clinically significant unless there is a 30% or greater reduction in air flow lasting for 10 seconds or longer with an associated 4% (or greater) desaturation in the person's oxygen levels, or if it results in arousal or fragmentation of sleep.

During a hypopnea there is airflow through throat but at a much reduced level, which leads to not getting enough oxygen. The apnea-hypopnea index or respiratory disturbance index (AHI) is an index of severity that combines apneas and hypopneas. Combining them both gives an overall severity of sleep apnea including sleep disruptions and oxygen desaturation (a low level of oxygen in the blood).

An apnea index or AI shows the average number of apneas per hour of sleep. A hypopnea index or HI shows the average number of hypopneas per hour of sleep. An apnea and hypopnea index or AHI shows the average number of apneas and hypopnea per hour of sleep. Some doctors use the term of respiratory disturbance index or RDI, instead of AHI. The apnea-hypopnea index, like the apnea index and hypopnea index, is calculated by dividing the number of apneas and hypopneas by the number of hours of sleep. AHI values are categorized as 5-15 mild, 15-30 moderate, and above 30 listed as Severe. Example: Apnea+Hypopnea divided by actual sleep time, and then multiply by 60. 200 Apnea 200 Hypopnea=400 Total Events; 420 Actual Sleep time (7 hours). Divide 400 by 420=0.95×60 (minutes per hour)=57 AHI (Severe OSA).

The physiological terms used to describe various types of breathing and associated with snoring or without obstructive sleep apnea breathing difficulties are: Eupnea—normal breathing, Apnea—absence of breathing, Bradypnea—decreased breathing rate, Dyspnea or shortness of breath—sensation of respiratory distress, Hyperaerationi/Hyperinflation—increased lung volume, Hyperpnea—fast and deep breathing, Hyperventilation—increased breathing that causes CO₂ loss, Hypopnea—slow and shallow breathing, Hypoventilation—decreased breathing that causes CO₂ gain, and Labored breathing—physical presentation of respiratory distress and Obstructive sleep apnea (OSA) is due to complete blockage of air to larynx due to mechanical soft tissue blockage (FIG. 3) by Naso-Oro-pharyngeal tissue.

There are Three Types of Sleep Apnea

1. Obstructive sleep apnea (OSA) is the common form of the condition when the tissues of the pharynx obstruct breathing during sleep. These pauses in breathing, called apneas (literally, “without breath”), usually last 20 to 40 seconds. There are more than 20 million suffer from OSA in US and its occurrence in adult population is estimated to be 3-4% in women and 6-7% in males. People who gain weight, develop obesity, craniofacial syndromes (mostly genetic), repair of cleft palpate, Down syndrome, small mandible etc. have a higher risk of developing obstructive sleep apnea than most individuals. The present invention is indented to treat these conditions.

2. Central sleep apnea is due to neurological condition as a result of a head injury, stroke, or nervous system disorder, and Heart failure. Patients with central sleep apnea should avoid using sedatives, narcotics, and alcohol.

3. Mixed sleep apnea is due to physical oropharyngeal airflow obstruction associated with central (CNS) etiology. It is a rare condition but is the most perilous form of sleep apnea and it is difficult to treat.

Symptoms of Obstructive Sleep Apnea are: Frequent cessation of breathing (apnea) during sleep; sleeping spouse or companion may notice repeated silences from your side of the bed then sudden awakenings to restart breathing with Choking or gasping during sleep to get air; Loud snoring; Waking up in a sweat during the night due to lack of oxygen and carbon dioxide build up; waking up restless in the morning after a night's sleep with or without Headaches, sore throat, or dry mouth in the mornings; daytime sleepiness including falling asleep at improper times, such as during driving, at work, at meeting and conferences and fatigue; mood changes such as irritability, anxiety and depression; trouble concentrating; forgetfulness decreased sex drive; unexplained weight gain; increased urination and/or nocturia; frequent heartburn, gastro-esophageal reflux disease (GERD); and heavy night sweats can also strike.

Studies by Lee et al has shown that the oxygen desaturation sleep events were detected in all patients with OSA but not in simple snorers (Lee C H, Mo J H, Kim B J, Kong I G, Yoon I Y, Chung S, Kim J H, Kim J W, Arch Otolaryngol Head Neck Surg. Evaluation of soft palate changes using sleep video fluoroscopy in patients with obstructive sleep apnea. 2009 ed; 135(2):168-72). When awake, inspiratory efforts increased the length and angle of the soft palate (SP) in patients with OSA but not in simple snorers. Elongation and angulations were greatest during desaturation sleep events and least during awake (FIGS. 1 and 3). In normal oxygenation events, changes in the soft palate (SP) were significantly larger in patients with OSA than in simple snorers (P<0.01 for SP length; P=0.03 for SP angle). These studies showed that the SP was considerably elongated and angulated in patients with OSA even when awake. Hence the treatment of snoring should be differentiated for the sake of treatment: 1. to prevent production of sound during sleep, 2. the obstructive sleep apnea with serious health consequences. The present invention is intended treatment of the both conditions.

There are no effective FDA approved drug treatments for obstructive sleep apnea. Nevertheless, a clinical trial of anti-depressants mirtazapine (Brand names: Remeron, Avanza and Zispin) has shown some promising results in the treatment of OSA; but it causes weight gains and sedation (“First Effective Drug For Sleep Disorder Identified”. ScienceDaily.com. June 2003). It is a tetracyclic antidepressant (TeCA) used primarily in the treatment of depression and as a hypnotic, antiemetic, and appetite stimulant, and for the treatment of anxiety. Mirtazapine is not a SSRI reuptake inhibitor. It disinhibits dopamine and norepinephrine activity in various parts of the brain, notably in the pleasure centers such as the ventral tegmental area (VTA), causing a pronounced antidepressant and anxiolytic response due to release of the neurotransmitters dopamine and norepinephrine. Beside its close analogues mianserin and setiptiline, mirtazapine is one of the small number of noradrenergic and specific serotonergic antidepressants (NaSSAs) that can be tried on OSA.

Serotonin uptake inhibitors (SSRI) such as fluoxetine, tryptophan, protriptyline; oral methylxanthine and theophylline (chemically similar to caffeine), amphetamines stimulants; to anti-narcoleptic medications such as modafinil are also tried. A course of anti-inflammatory steroids such as prednisone (or another glucocorticoid drug) is given to reduce the lymphoid tissue of the naso-oropharyngeal air passages if enlargement of the lymphoid tissue is found and the allergic conditions are suspected.

A basic treatment for snoring with or without obstructive sleep apnea involves having the patient sleep in the prone position or on his/her side. Sometimes this is stimulated by sewing an object into the back of the snorer's clothes. In an obese patient, treatment includes weight loss. Along with these treatments the recommendation that the patient avoid use of CNS depressing drugs, cigarettes, or alcohol prior to bedtime to prevent or reduce the loss of oropharyngeal muscle tone.

Air passage obstruction due to enlarged tonsils or adenoids may need to be removed. In some cases surgical repair of a deviated nasal septum has been shown to improve snoring. Snoring can also be due to genetics with some being predisposed towards an anatomical narrowing across the nasal-oral pharynx. A reduced nasopharyngeal passageway may also be caused by a lack of muscle tone. Other anatomical conditions contributing to the narrowing of the nasal pharyngeal passageway include choanal atresia, chrono polyp, nasal septal deviation, nasal and pharyngeal cysts, macroglossia, retrognathia, and micrognathia. (Leung et al, “The ABZzzz's of Snoring”, Post Graduate Medicine; Sep. 1, 1992).

Snoring and OSA might be aggravated by alcohol drinks or drugs (such as tranquilizers, hypnotic, sleeping pills, and antihistamines) taken prior to bedtime. Smoking is also held responsible for snoring, since cigarettes may irritate the mucus membranes of the upper airway and oropharynx; causing swelling and increased mucus production. Where snoring is caused by nasal allergy or an upper respiratory tract infection, these conditions may be treated with antiallergenic treatment. (Douglas—N.J., “The Sleep Apnoea/Hypopnoea Syndrome And Snoring”, British Medical journal, 1993, Vol. 306:1057-60; Leung et al, “The ABZzzz's of Snoring” Post Graduate Medicine (Sep. 1, 1992).

Anti-snoring and anti OSA devices are shown to be effective when they pull or hold the mandible (lower jaw) forward and upward; and elevate the tongue as the muscles of the mandible relax, so that the tongue does not occlude the air passageway drifting inferiorly and posteriorly while sleeping so as to prevent the passage of air (FIG. 1). Most anti-snoring devices accomplish this task by moving the lower jaw forward and holding that position against a rigid upper dental component, which is fixed to the upper teeth in the immobile maxilla and to the lower teeth in the mandible. The disadvantages in using the above prior art devices, is that they require expert qualified licensed lab services for fitting of the anti-snoring device to the user's mouth.

Such devices could cause permanent irremediable changes in the bite of the user and permanently alter the jaw position and it requires a dentist to closely monitor anti-snoring device fitting. There is a need for an anti-snoring device that does not rigidly bind to the dental structures of the user's mouth and that does not require professional supervision or assistance in its fabrication, monitoring of the dental bite changes, and mandibular changes. In addition, the anti-snoring device should not pit the lower jaw against the upper jaw. These devices do not include an intra oral dental overlay to support the tongue against the palate and keep the palate of the user's mouth from reverberating (snoring) during mouth breathing. The present invention overcomes these draw backs.

Snoring and OSA can sometimes be managed by the use of a positive pressure generator and facemask. In this procedure: A mask that covers your nose and mouth or just nose or mouth. The standard method is Continuous Positive Airway Pressure (CPAP) treatment, which requires the patient to wear a mask through which air is blown into the nostrils to keep the airway open. Patient compliance is poor due to discomfort and side effects. These machines pump air through a hose and nose/mouth face mask to keep air passages clear. CPAP pneumatically splints the upper airway. Use of the devices, can cause the subject to become non complaint due to difficulty in its use due to discomfort problems during sleep. Problems that may occur with CPAP include: restless sleep, dryness of nose, throat, and nasopharyngeal tract, cough, excessive dreaming during early use, nasal congestion, runny nose, sneezing, irritation of the eyes and the skin on the face, abdominal bloating, and leaks around the mask because it does not fit properly.

You may be able to limit or stop some of the side effects: your doctor may be able to adjust your CPAP to reduce or eliminate problems; make sure the mask or nasal prongs fit you properly; air should not leak around the mask; use a humidifier or a corticosteroid nasal spray medicine to reduce nasal congestion, irritation, and drainage.

User may want to talk to a doctor about trying a CPAP machine that will help to reduce discomfort caused by too much constant pressure in the user's nose. If this does not improve discomfort, ask your physician about trying a bi-level positive airway pressure machine (BiPAP-VPAP or variable positive airway pressure), which uses a different air pressure when you breathe in then when you breathe out. BiPAP may work better than standard CPAP for treating obstructive sleep apnea in people who have heart failure. BiPAP machines are more expensive than CPAP machines.

When one is using CPAP, the person need to see their doctor or sleep specialist regularly and might even need more sleep studies to adjust the CPAP machine and check whether the treatment is working. The sleep studies and the CPAP machines are expensive and one can rent a CPAP machine before one buys it. The most common problem with CPAP is lack of compliance meaning that people do not use the machine every night because it is uncomfortable, and take it off as they sleep, feel sleepy next day due to repeated interruption during sleep

A more recent treatment option to obstructive sleep apnea includes the implantation of rigid inserts in the soft palate to provide structural support, is both invasive and is only effective for mild to moderate cases of obstructive sleep apnea. Alternative treatments are even more invasive and drastic, including tracheostomy, genioglossus advancement or stimulator, hyoid suspension, tongue reposition, and tissue ablation (somnoplasty or uvulopalatopharyngoplasty (UPPP)).

If all else fails, sleep apnea can be effectively treated by maxillomandibular advancement. It is a complex operation in which the maxilla holding the upper teeth and the mandible holding the lower teeth are surgically cut and moved so that the lower part of your face is moved forward approximately 12 millimeters. In this complex surgical procedure, the airway in the back of the throat is expanded and the obstructive sleep apnea alleviate. This undertaking is advised only for disabling obstructive sleep apnea patients in whom other treatments have failed and the OSA is life threatening. Reduction of parts of the soft palate, laser-assisted uvulopalatoplasty, reduction of the tongue base either with laser excision or radiofrequency ablation, Genioglossus Advancement, Hyoid Suspension in which the hyoid bone in the neck are attempted to treat this condition. In rare intractable cases, tracheostomy is the only effective treatment for sleep apnea.

Due to many associated disadvantages, complications and high failure rate, these tissue ablation methods and radicle surgeries need to be considered only in desperate patients. Other options for treating snoring are found with surgical techniques such as removal of enlarged adenoids, tonsils, and host of other therapies are recommended. Surgical removal of the uvula, distal portion of the soft palate, the anterior tonsillar pillars, and the redundant lateral pharyngeal wall mucosa is said to increase the size of the air passageway allowing unobstructed movement of air through the pharynx. Rates of success of the uvulopalatopharyngoplasty are reported to be in a range from 15% to 65%. (Douglas, “The Sleep Apnoea/Hypopnoea Syndrome And Snoring”, British Medical journal, 1993, Vol. 306:1057-60). In some instances, surgical repair of a deviated nasal septum has been shown to improve snoring but not OSA.

Snoring and obstructive sleep apnea results in exhaustion resulting from lack of sleep and interfering at work as well as while driving is a problem. Obstructive sleep apnea causes high blood pressure, depression, irregular heart rhythms, heart failure, coronary artery disease, and stroke. If you are overweight, bariatric surgery may help you lose weight, which may improve sleep apnea.

Snoring and obstructive sleep apnea patients with decreased pulmonary function such as emphysema, asthma, and chronic obstructive lung diseases (COPD) have been shown to suffer from severe apnea. Cessation of breathing during snoring, or obstructive sleep apnea results in lack of oxygen due to an obstructed nasopharyngeal passageway deprives the body of sufficient oxygen so that the oxygen de saturation arises. Lack of oxygen may cause the brain to rouse the sleeper just sufficient to take a breath without fully awaking. Since this may happen hundreds of times a night, the snorer, and OSA patients do not get sufficient sleep. Moreover, being aroused from deep REM sleep on a repetitive basis increase heart rate and blood pressure with associated increase the risk of heart attack and stroke. Furthermore, due to narcolepsy resulting from exhaustion can cause a lack of attention for the snorer and OSA sufferers during waking hour's causing drop in productivity and accident prone at work and driving.

U.S. Pat. No. 5,569,679 discloses the use of nasal solution 10%-16% of methylsulfonylmethane (MSM) drops for the treatment of anti-snoring method. It is a nasal spray, too simplistic to a complicated anatomically related snoring with or without obstructive sleep apnea whose pathophysiology is not in the nose.

U.S. Pat. No. 5,921,241 discloses an anti-snoring device including a moldable dental overlay for covering the lower teeth of the user and for maintaining the tongue in contact with the palate to prevent air flow from causing the palate to reverberate during mouth breathing.
U.S. Patent Application Publication Number: us 2004/0153127 Al invention provides electrical stimulation that causes the oropharyngeal muscles to contract during sleep using one or more micro stimulators injected into or near these muscles or the nerves which innervate them.
U.S. Patent Application Publication Number: US 2007/0233276 A1 describes the method and apparatus include placing a tissue contractor within the tongue tissue. This is invasive procedure and may create discomfort and complication after surgery.
U.S. Pat. No. 6,418,933 B1 discloses an anti-snoring device has maxillary and mandibular bite forms with outwardly extending pivots which are mounted to the bite forms by frameworks which are at least partially embedded in the bite forms.
U.S. Pat. No. 5,499,633 shows two bite forms which may be joined so that the user's mandible projects forwardly of its normal position in order to reduce snoring.
U.S. Patent Application Publication Number: 2005/0178392 A1 discloses a small piece of cloth tape or other porous hypo allergenic material with a hypo allergenic adhesive on the back is affixed to the lips before sleeping. May not be effective in preventing the vibration of the soft palate and snoring with or without obstructive sleep apnea.
U.S. Pat. No. 7,016,736 B2 discloses a submental electrical stimulation of the supra hyoid muscles at the floor of the mouth, does not address the snoring due to vibration of the soft palate and uvula.

Numerous management techniques have been described, and none of these treatments have proved adequate and most of the therapies are inadequate to treat snoring and obstructive sleep apnea. Surgery for the condition is fraught with fear and complications besides high cost and high rate of failure. Hence, the snoring without or without obstructive sleep apnea remains a serious health problem. With increasing obesity, snoring with or without obstructive sleep apnea is increasing in the general population along with type II diabetes. Accordingly, there has been a need for improved management techniques to reduce or eliminate snoring and obstructive sleep apnea using simple and safe methods. The devices in the present invention are designed to be used to treat snoring and obstructive sleep apnea with minimum or no complications, least disadvantages, and highest compliance.

### SUMMARY OF THE INVENTION

A snoring and obstructive sleep apnea prevention and treatment device has an oral device portion with a centrally disposed plate portion. The plate portion has a suction cup on its tongue facing side which helps hold it in place and to keep the tongue from moving back towards the throat, thus preventing snoring and sleep apnea. A hood portion fits around the tip of the tongue further preventing the tongue from sliding back into a user's throat. In one embodiment, an inflatable balloon portion is provided to prevent soft tissue from contacting portions of the tongue. In another embodiment, a plate is used in place of the balloons. Additionally, oxygen may be supplied to a posterior portion of the device. Dental adhesives such as denture adhesive may be used to further restrict movement.

It is an object of the present invention is to provide a new, useful, simple, and effective device for the prevention and treatment of snoring and obstructive sleep apnea (OSA).

Another object of the present invention is to provide a safe and effective treatment device which can be self-fitted or inserted in the mouth by a snorer and obstructive sleep apnea patients before going to sleep.

A further object of the present invention is to provide a safe and effective treatment device inexpensively to prevent snoring and obstructive sleep apnea.

An important function of this invention is in allowing unobstructed movement of the air through the nose, mouth, and pharynx to the larynx to prevent hypoxia, snoring, and obstructive sleep apnea

Yet another purpose of the present invention is to provide an inexpensive method to reduce the incidence of snoring and obstructive sleep apnea that is safe and effective.

Yet another goal of the present invention is to provide an invention relates to an apparatus inserted into oral cavity which extends all the way to the posterior aspect of the tongue to prevent snoring and obstructive sleep apnea.

Yet another goal of the present invention where in the device is in contact of the tongue and soft palate and prevent flaccid tissue fall back during sleep so as to cause snoring and OSA.

The apparatus includes a tongue glove with silicone balloon or palate shelf which comes in contact with the tongue, soft palate and uvula and prevents the soft palate and uvula to vibrate and prevents backwards moving of the flaccid tongue during sleep, thus preventing snoring and obstructive sleep apnea.

The apparatus is placed on the dorsal surface of the tongue liken to a mitten in the middle of the tongue.

The shape of the apparatus conforms to the shape of the tongue, slightly convex upwards so as to place it easily and fits snugly under the palate.

The convex surface the apparatus has a convex metal shelf or projection (palate shelf) which prevents the soft palate coming down, and be in touch with the tongue so as to produce the sound of snoring associated with or without obstructive sleep apnea.

This invention also provides a soft palate and uvula balloon, when blown, will prevent the soft palate and uvula vibrating contributing to snoring.

This invention provides a palate and uvula balloon, with bulge of the balloon placed against the back of the tongue. When this balloon is inflated, it will prevent the soft palate and uvula coming in contact with the back of the tongue and prevents the tongue moving backwards and block the oropharynx so as to cause snoring and obstructive sleep apnea.

The central hard plate of the device has suction cups, which are attached to hard palate by simple vacuum pressure or dental adhesive made to stick to hard palate. The undersurface of the device also has suction cup which can be attached to the dorsal surface of the tongue with adhesive or air vacuum du suction cups. When the device is in place with attachment to the hard palate and dorsal surface of the tongue, prevents the tongue drifting backwards when it becomes flaccid during sleeping, so as to cause obstructive sleep apnea thus prevent the obstructive sleep apnea.

This novel invention apparatus has a quadrangular plastic or thin metal plate, and in the middle of the device which holds the suction adhesive cups and the balloon firmly in place during use.

The device has two “V” shaped receivers made of metal or plastic, silicone, other synthetic or combinations of composite material so as to fit the upper and lower incisor teeth (incisor teeth receptors) which can be adjusted to the comfortable levels to pull the lower jaw forwards on the fixed upper jaw to prevent the backward movement of the tongue during sleep.

This inventive device is provided with oxygen canula so as to supply oxygen from the oxygen tank or oxygen generator to provide supplementary oxygen at laryngeal inlet as needed to prevent hypoxia.

According to the present invention, the device can be provided with injection/inflation port and canula to deliver any therapeutic agents and local anesthetics to reduce the sensitivity of the oropharyngeal passages when the device is in place in the mouth before sleeping.

An addition, the anti-snoring and anti-obstructive sleep apnea device is provided with submental electrical stimulator to stimulate supra hyoid muscles at the floor of mouth which includes electrodes to be arranged below the tongue and above the mucous membrane covering the floor of mouth. The electrical stimulation increases the tone of the tongue muscles and prevents the flaccidity that takes place during sleep; thus prevents obstructive sleep apnea.

It is another intention of this invention is to wear the device without interruption of sleep at night.

Other features and advantages of the instant invention will become apparent from the following description of the invention which refers to the accompanying drawings.

### BRIEF DESCRIPTION OF THE DRAWINGS

FIG. 1 is a diagrammatic presentation of the air way during awake and sleeping.

FIG. 2 is a diagrammatic presentation of the air way with soft palate and tongue partially obstructing the airway resulting in snoring.

FIG. 3 is a diagrammatic presentation of the air way with soft palate and tongue completely obstructing the airway resulting in obstructive sleeping apnea (OSA).

FIG. 4 is a diagram showing dorsal surface of the device used for stopping snoring and obstructive sleep apnea.

FIG. 5 is a diagram showing the ventral surface of device used for stopping snoring and obstructive sleep apnea.

FIG. 6 is a diagram showing side view of the device used during sleep to stop snoring and sleep placed in the mouth.

FIG. 7 is a diagram showing the device to stop snoring.

FIG. 8 is a diagram showing the device to stop snoring on the dorsal surface of the tongue.

FIG. 9 is a diagram showing all the embodiments of this inventive the device to stop snoring and obstructive sleep apnea.

FIG. 10 is a diagram showing the device to stop snoring with metal or plastic shelf placed in the mouth.

FIG. 11 is a diagram showing the device with balloon to stop snoring.

FIG. 12 is a diagram showing the device in the mouth with suction cups to stop snoring and obstructive sleep apnea.

FIG. 13 is a diagram showing the anti-snoring and obstructive sleep apnea device expander and insertion forceps to facilitate placing the device on the protruding tongue.

FIG. 14 is a diagram showing the anti-snoring balloon with a ring like expansion which allows the air to flow from the nose to the larynx without pharyngeal obstruction.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

According to the present invention, the snoring and obstructive sleep apnea patients (OSA) are treated by recognizing patient snoring with or without obstructive sleep apnea attributable at least in part due to the vibration of the soft palate during inspiration and movement of a base of a tongue of said patient toward a pharyngeal wall of said patient so as to cause obstruction to free air movement from external source through the nose to larynx.

The method includes detecting a region in the tongue extending from a mandible to the base of the tongue; preventing the muscular tissue of the tongue moving back towards the pharynx; and vibration of the soft palate resulting in the snoring and obstructive sleep apnea. The present invention prevents the tongue of the patient from obstructing the air passage. The proximal portion of the device is secured to incisor teeth by use of incisors teeth receptacles or pockets of the jaw bone of the patient with rest of the apparatus inserted along the dorsal surface of the tongue at the same time holding the soft palate in such position by palatine shelf or balloon so that it does not vibrate coming in contact with the tongue or participate in snoring and obstructive sleep apnea.

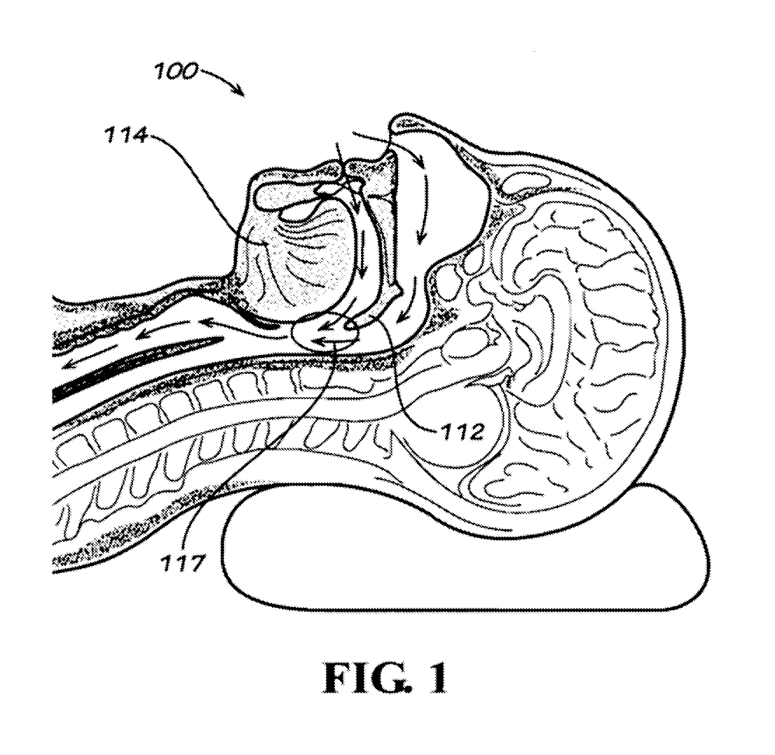

FIG. 1 is the diagrammatic presentation of the normal air way 100 with soft palate 112 and tongue 114 not obstructing the airway passages 117 allowing the free flow of air from the mouth and the nose as the person sleeps on pillow. The air flow does not produce a physical force like a narrow air stream, hence does not produce snoring nor cause obstructive sleep apnea.

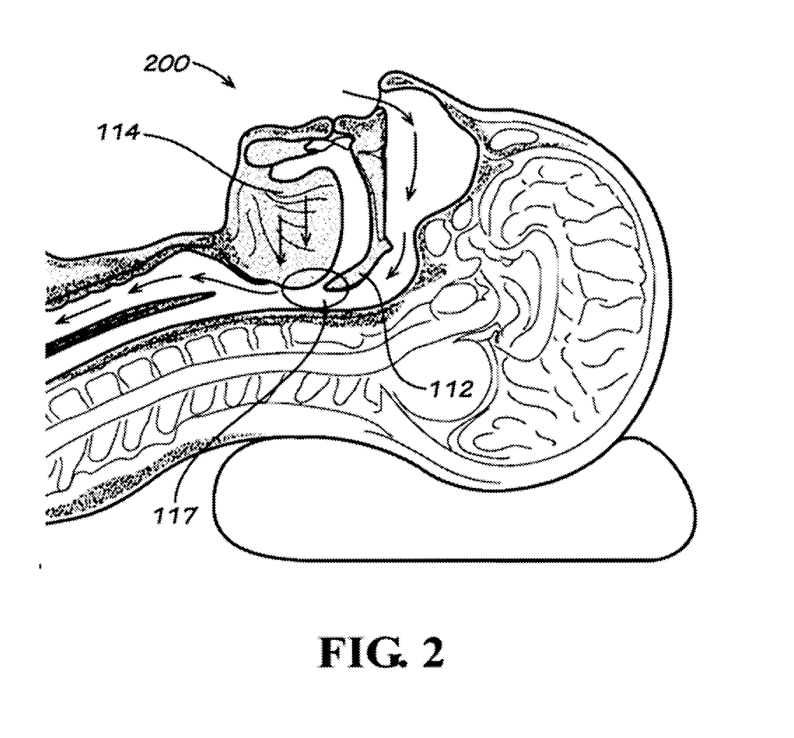

FIG. 2 is the diagrammatic presentation of the air way 200 with soft palate 112 and tongue 114 partially obstructing the airway 117 not allowing the free flow of air from the mouth and the nose as the person sleeps. The air flows in a narrow stream through the air passages 117 vibrating the soft palate 112 and soft tissue around the tongue 114 producing snoring as one sleeps. This is due to relaxation of soft palate 112 and tongue 114 becoming flaccid and falling back so as to create forcible air stream like venturi effect especially the soft palate 112 which does come in contact with narrow stream force of air so as to produce sound as one falls asleep.

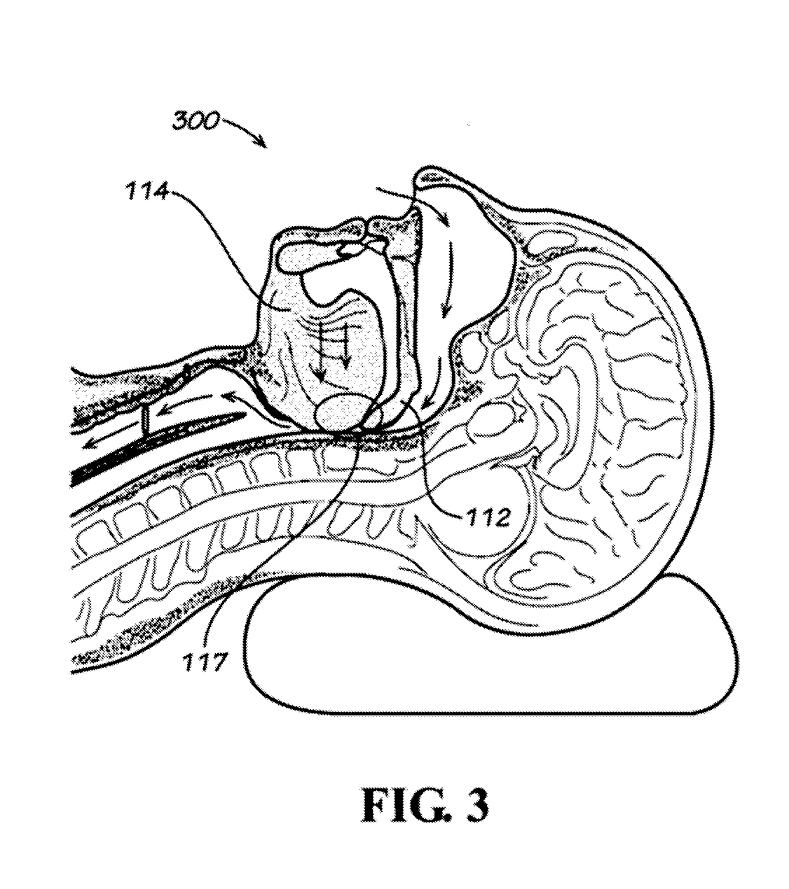

FIG. 3 is the diagrammatic presentation of the air way 300 with soft palate 112, tongue 114 completely obstructing the airway 117 by not allowing the free flow of air from the mouth, and the nose to the larynx through the oropharyngeal passageway as person sleeps. This is due to relaxation of soft palate 112 and tongue 114 becoming flaccid and falling back on the wall of the oropharynx 117 so as to create obstruction to passage of air resulting in obstructive sleep apnea (OSA).

When the person becomes aware due to central nervous system activation, can resulting in partial opening of air way allowing the air stream which causes vibration soft tissue of the oropharynx, especially the soft palate 112 which come in contact with narrow stream force of air so as to produce snoring sound as one falls asleep. The air flows in a narrow stream of air or complete obstruction to air passage 117 can result in vibrating the soft palate 112, and the soft tissue around the tongue 114 producing snoring and complete obstructive sleep apnea.

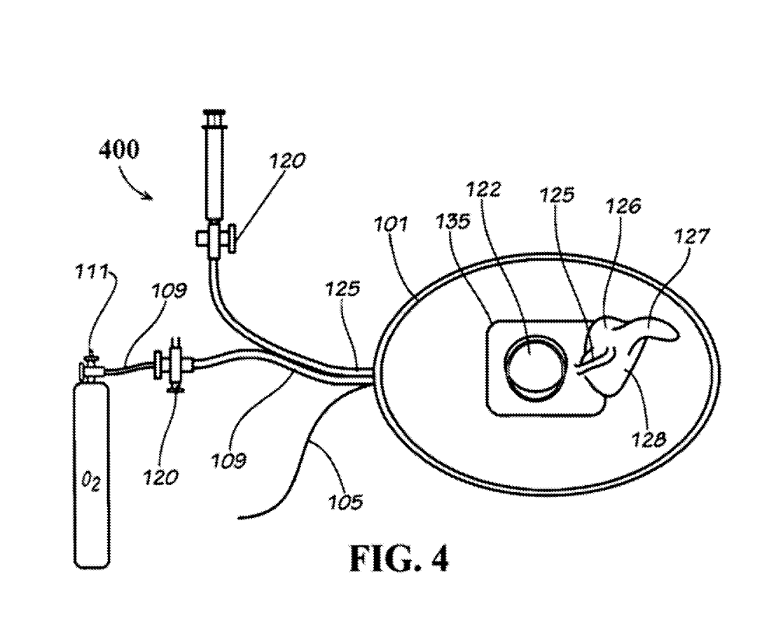

The present invention as shown in the FIGS. 4 through 12 and 14 will prevent the obstruction to the air flow (FIG. 3), prevent the soft palate-uvula vibrating (FIG. 2), and stop tongue falling back as described in the diagrams by keeping the air way open physiologically and mechanically; thus providing the effective method for the treatment of snoring and obstructive sleep apnea.

FIG. 4 is a diagrammatic presentation 400 showing the details of the invention for prevention and treatment of snoring and obstructive sleep apnea. The device 101 is made of soft plastic or other synthetic or natural composite material like hand gloves in the shape of the tongue which fits on entire dorsal surface of the tongue from the tip of the tongue and margin of the tongue snugly. When positioned on the tongue, the device covers the free part of the tip and edges of the tongue like glove or hood. In the center, the device has a slightly hard malleable plastic or thin metal plate 135 and the center contains a suction cup 122. The distal part of this plate has a balloon, which can be inflated by tubing 125 and form a canula connected outside with inflatable syringe 120. The syringe connected to the tubing 125 inside the balloon that can be expanded by air, saline or gelatinous semi liquid material.

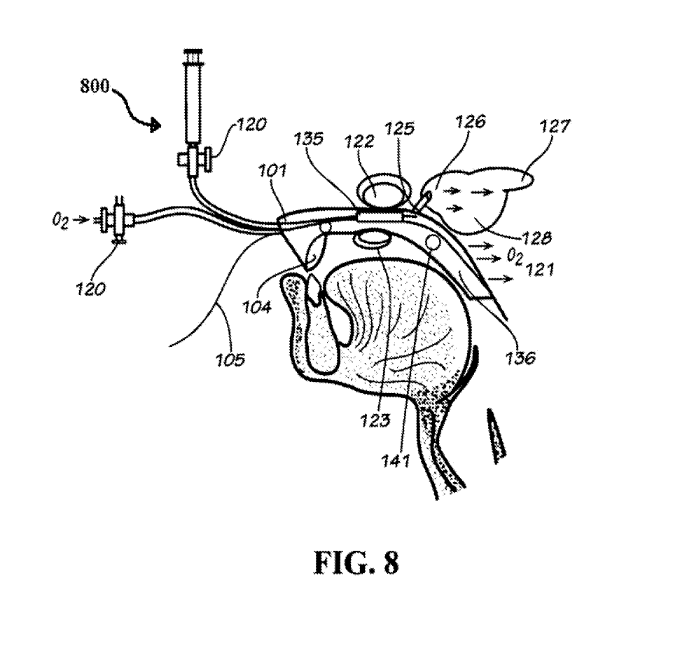

When filled, the balloon assumes three bulging, the bulging 126 pushes the soft palate upwards, the uvular extension 127 pushes the uvula upwards and backwards, and tongue bulging 128 pushes the tongue downwards and forwards and holds it in position and will not allow it to move or drift backwards so as to cause snoring and obstructive sleep apnea. When in place with the balloon blown will push the soft palate and uvula upwards and the base of the tongue downwards and forwards as shown in FIGS. 8, 10, 11, and 12.

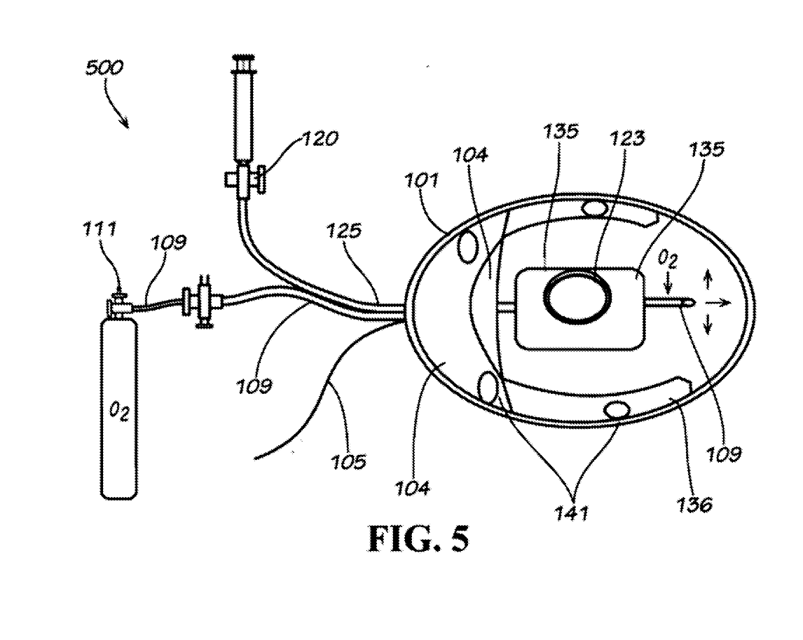

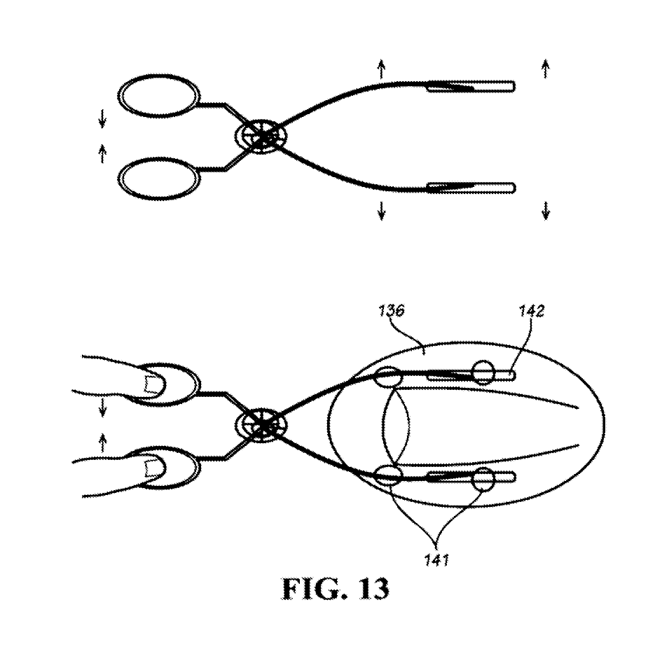

The suction cup when pressed against the hard palate holds the tongue in position without allowing it to drift backwards (posteriorly) and inferiorly and thus prevents its blocking effect to produce obstructive sleep apnea. The suction cup can be adhered to the hard palate by use of denture adhesive or by creating vacuum in the suction cups by pressing the tongue against the hard palate. This device is connected by canula to oxygen supply tank 111 by the way of tubing 109 with a stopcock 120. It has thin thread safety portion 105 attached to the tip of the device to prevent it being swallowed or aspirated accidentally while asleep. The device is provided with stretching placement forceps shown in FIG. 13 which keeps it open and facilitates its placement on the surface of the tongue with ease. Dental adhesive commonly used to hold the dentures can be applied to the suction cup 122 and 123 (in FIG. 5) to attach the device to the hard palate and dorsal surface of the tongue.

FIG. 5 is the diagrammatic presentation 500 showing the details of the invention for treatment of snoring, and obstructive sleep apnea. The device 101 is similar in all the explanations as described in FIG. 4. It shows the under or ventral surface of the device which covers the tip, sides and dorsal surface of the tongue and holds fit snugly. It has the ventral hood or cap like enclosure 104 which covers the free tip and its undersurface of the tongue. From the edges of surface of the dorsal aspect of the device; there is small skirt 136 extending from the ventral hood 104 which covers the free margin of the tongue all the way to the root of the tongue.

In the middle, it has thick malleable plastic plate 135 from which the suction cup 123 facing the dorsal surface of the tongue is located in the middle of the hard plate. At the end of the undersurface is the canula 109 which delivers oxygen close to the laryngeal opening from the external oxygen 111 source. When applied on the tongue, the device is held in place by the hood or cap 104 and the suction effect of the suction cup which can also hold device in place on the tongue. The device is held in place with dental adhesive or vacuum effect of suction cups. It is provided with 4 holes 141 in the skirt 136 for inserting of the tongs or forceps tips as shown in FIG. 13 for opening the device and placing it on the tongue with ease before going to sleep.

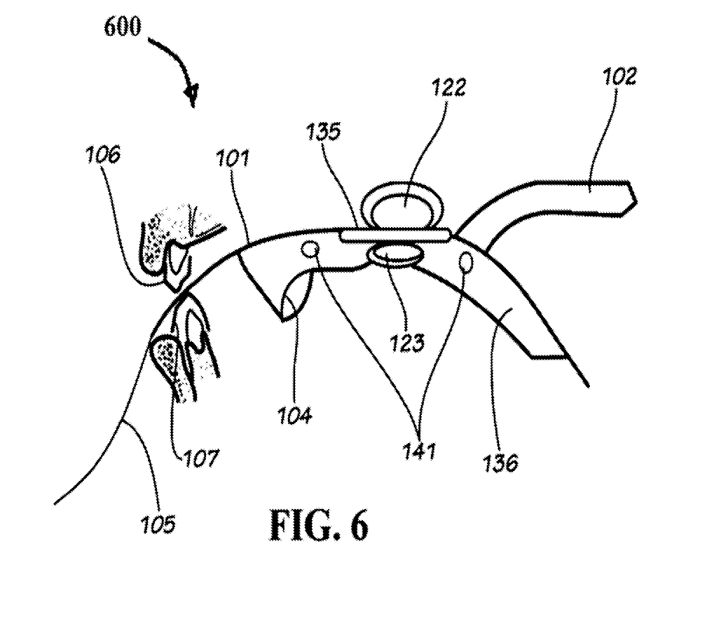

FIG. 6 is the diagrammatic presentation 600 showing the details of the invention for treatment of snoring, and obstructive sleep apnea. It is the side view of the device 101 encompasses all the embodiments described in FIGS. 4 and 5. The device 101 has a cap or hood 104 which covers the free margin of the tip of the tongue like a cap and short skirt 136 which covers the sides and free margins of the tongue all the way to its root. Instead of balloon as described in FIG. 4, it contains a flat shelf 102 which hold the soft palate and uvula lifted up to prevent the sound during sleep.

It has dorsal 122 and ventral 123 suction cups attached to the plastic plate 135 which attaches the device to the hard palate and dorsal surface of the tongue. The device is provided with upper 106 and lowers 107 incisor teeth sockets pocket which will facilitate the holding of the lower jaw (mandible) moving forwards over the fixed upper jaw. It also has a string 105 attached at the front end to prevent it being accidentally swallowed or aspirated during sleep. The device also has 4 holes 141 in the skirt 136 for inserting of the tongs tips or forceps for opening the device and placing it on the dorsal aspect, the tip and the sides of the tongue with ease before going to sleep.

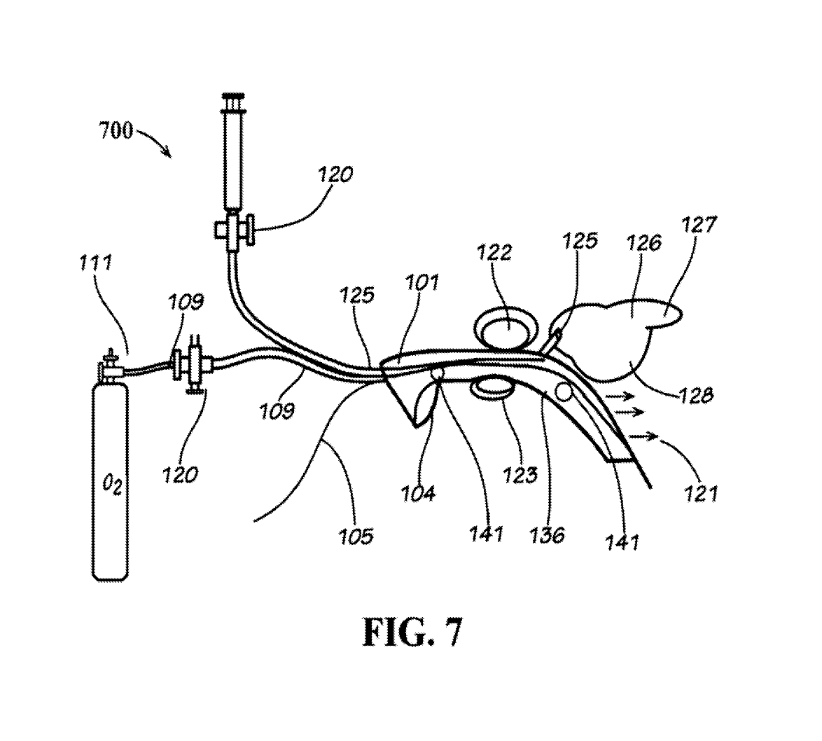

FIG. 7 is the diagrammatic presentation 700 showing the details of the invention for treatment of snoring, and obstructive sleep apnea as described in FIGS. 4 and 5. The device 101 has hood or cap 104 which covers the free end of the tongue to hold the device on the tongue. It has skirt 136 on the side to cover the free lateral edges of the tongue. The device in the middle has dorsal 122 and ventral 123 suction cups when pressed will hold the device firmly attached to the hard palate and dorsal surface of the tongue. From the back of the device located on back of the device is balloon when inflated has 3 bulges.

The balloon bulge 126 pushes the soft palate up, the bulge 127 holds the uvula in place preventing the production of sound of snoring during sleep and prevent the soft palate coming in contact with the posterior dorsal aspect of the tongue and oral pharynx. The tongue bulge 128 pushes the tongue from the back surface forwards and downwards and prevents the tongue moving back on the oral pharyngeal wall when it becomes flaccid during sleep.

It also has oxygen (O₂) exits 121 delivered on the back of the tongue close to the proximity of the laryngeal opening and provides supplemental oxygen from the external source 111 through the oxygen delivery tubing 109. It is provided with the stop cock 120 also to block the delivery of oxygen if needed. The device has 4 holes 141 in the skirt 136 for inserting of the tongs tips or forceps for opening the device and placing it on the tongue with ease before going to sleep.

FIG. 8 is the diagrammatic presentation 800 showing the details of the invention for treatment of snoring and obstructive sleep apnea. The device 101 is the same as explained in the FIG. 7 except it is placed on the tongue and shows how it is placed on the tongue behind the mandible and in front of the pharyngeal airway. The device has 4 holes 141 in the skirt 136 for inserting of the tongs tips or forceps for opening the device and placing it on the tongue with ease before going to sleep. The device is made to stay on the tongue by pressing the suction cups 122 and 123 against the hard palate to dorsal surface of the tongue to create negative pressure and hold it in position with dental adhesives placed on the suction cups.

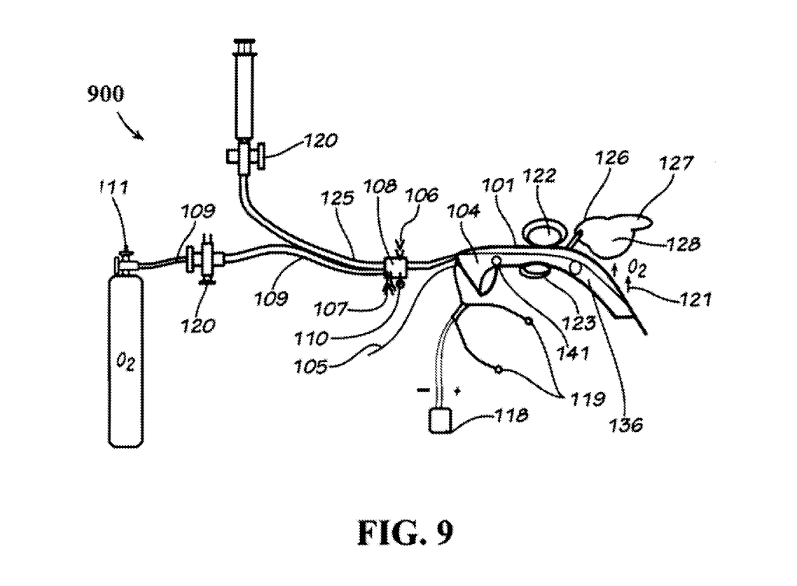

FIG. 9 is the diagrammatic presentation 900 showing the details of the invention for treatment of snoring, and obstructive sleep apnea as described in FIGS. 4, 6, 7, and 8. The device 101 encompasses complete embodiments of the device used to prevent snoring and obstructive sleep apnea. In addition to the above described embodiments explained in the above diagrams; it has additional embodiment incorporated namely submental supra hyoid muscle stimulator 119 connected to electrical battery source which has milliamp delivery adjuster 118.

The electrical stimulator goes under the free margin of the tip of the tongue and side of the tongue encircling the root of the tongue. This electrical simulator increases the tone of the muscles of the root of the tongue and prevents it drifting downwards and backwards due to relaxation of these muscles during sleep, thus prevents the snoring with or without obstructive sleep apnea. It is provided with supplemental oxygen O₂ delivery system 111 connected to tubing 109 and extending all the way to the back of the device 121 to facilitate its delivery close to the laryngeal inlet to enter the lungs during inspiration.

The inflated balloon prevents the tongue sliding back to the pharyngeal wall, does not allow the soft palate and uvula coming in contact with the tongue and pharyngeal wall, and thus prevent the snoring with or without obstructive sleep apnea. It also has adjustable sliding attachment 108 with tightening screws 110; which has incisor teeth sockets 106 and 107 to move the lower jaw forwards on the fixed upper jaw. This additional attachment helps to prevent the movement of the flaccid tongue backwards towards the oropharyngeal wall. The device also has 4 holes 141 in the skirt 136 for inserting the tongs tips or forceps for opening the device and placing it on the tongue with ease before going to sleep.

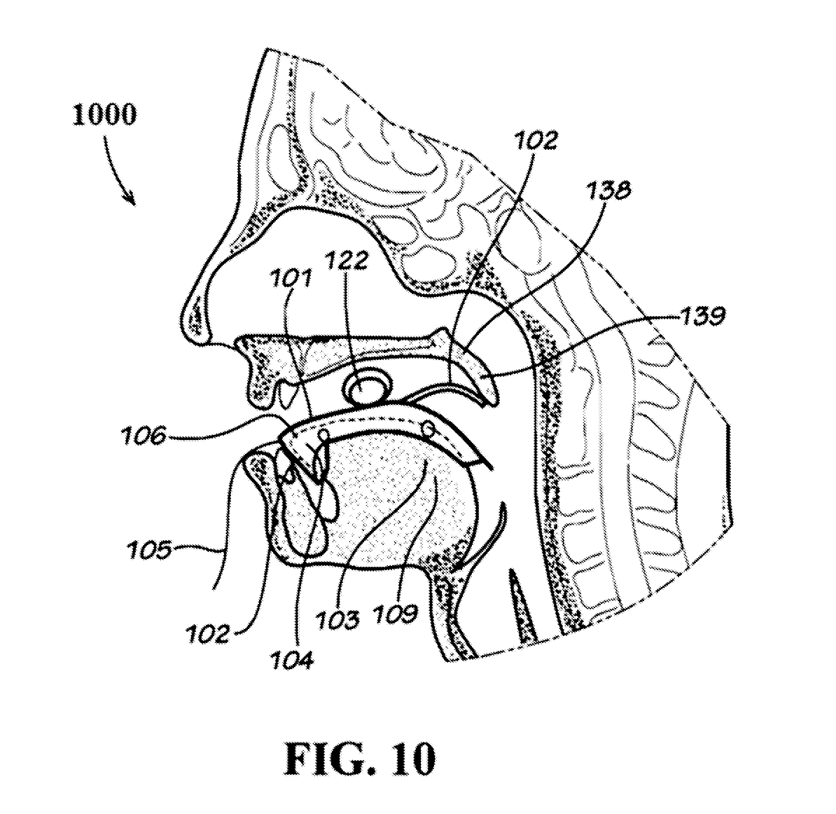

FIG. 10 is a diagrammatic presentation 1000 showing the details of the invention for treatment of snoring, and obstructive sleep apnea placed in the mouth in operation. The device 101 is similar to the one explained in FIG. 6 placed in the mouth. Note the palate shelf 102 lifting the soft palate 138 and uvula 139 and prevent the snoring during sleep. It is also provided with dorsal 122 and ventral surface suction cups to hold it in place during sleep when in use. It is also provided with thread 105 placed outside the mouth to prevent the device 101 accidently swallowed or aspirated during sleep. The device also has 4 holes 141 in the skirt 136 for inserting of the tongs tips or forceps for opening the device and placing it on the tongue with ease before going to sleep.

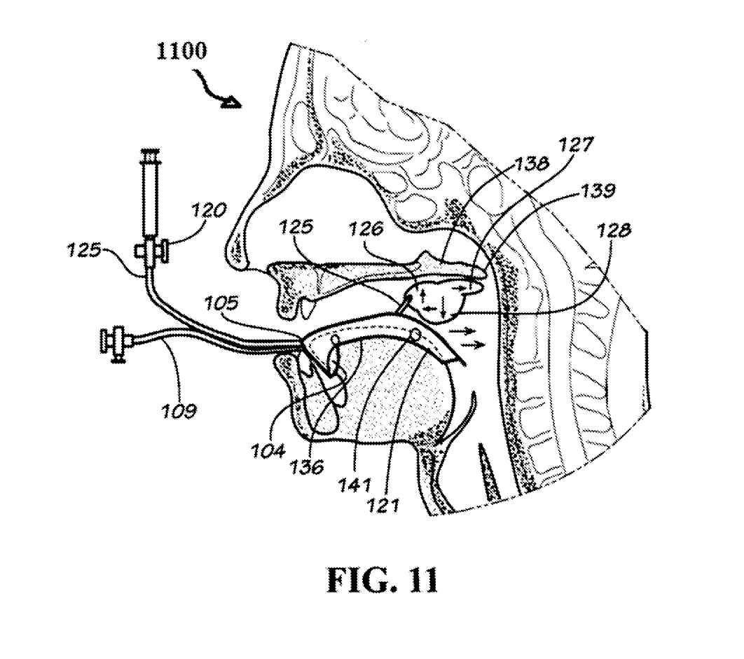

FIG. 11 is the diagrammatic presentation 1100 showing the details of the invention for treatment of snoring, and obstructive sleep apnea device in the oral cavity in operation. It is another type of anti-snoring and anti-obstructive sleep apnea device 101 which has only balloon on the back of the device which can be inflated by canula 125 from external source 125 syringe using air or gel or liquid. The device does not have suction vacuum cups. The device is placed on the tongue and when in use the inflated balloon bulge 126 lifts the soft palate, the balloon projection 127 pushes up the uvula and prevents it coming in contact with the back of the tongue, and the balloon projection 128 pushes the tongue forwards and downwards and holds it in its root thus preventing it moving inferiorly and posteriorly towards the oropharynx during its flaccid state for the duration of the sleep and thus prevents the obstructive sleep apnea and snoring. The tongue hood or cap 104 holds the device in position without displacement or sliding backwards during sleep. It is also provided with string 105 attached to the device to prevent accidental swallowing or aspiration during sleep. The device has 4 holes 141 on the skirt 136 for inserting of the tongs tips or forceps for opening the device and placing it on the tongue with ease before going to sleep.

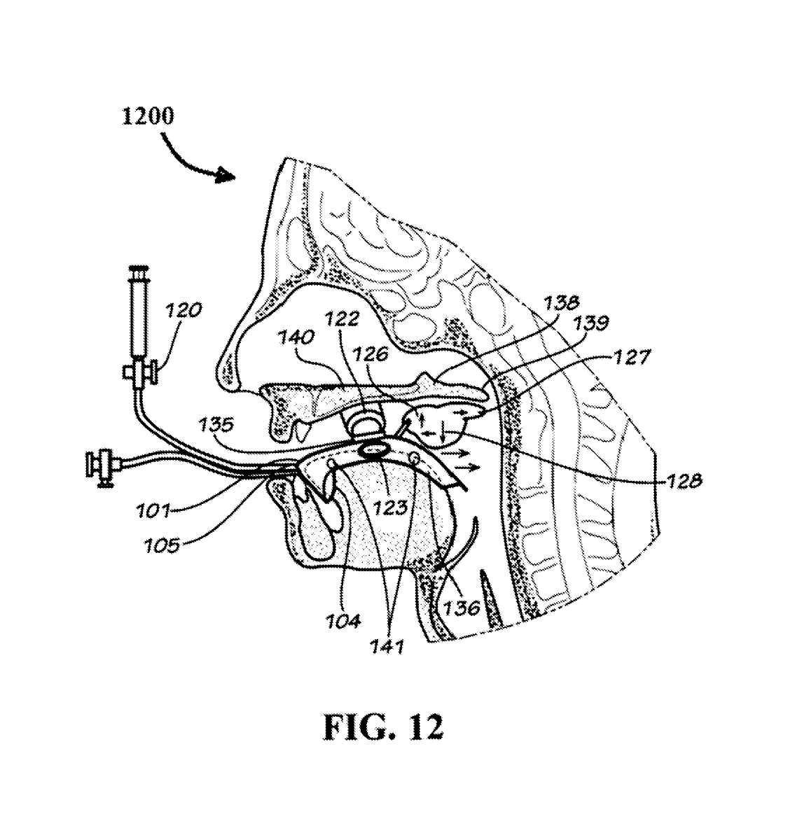

FIG. 12 is the diagrammatic presentation 1200 showing the details of the invention for treatment of snoring and obstructive sleep apnea. The device 101 is similar to device explained in FIG. 11 and it show in addition other embodiments. The balloon has its soft palate 126, uvula 127, tongue 128 bulges, and it has suctions cups 122 that can be pressed against the hard palate 140 and the dorsal aspect of the tongue 123. These vacuum creating suction cups are attached to the rigid plastic plate 135 so as to hold the suctions cups in place and can be easily put to action as user of the device by pressure application against the plastic plate 135 by moving the tongue against the hard palate 140.

The balloon pushes the soft palate, uvula and tongue 126, 127, 128 plays an important role in prevention of snoring and obstructive sleep apnea. Attaching the device to the hard palate 140 and dorsal surface of the tongue with its tip hood or cap 104 also plays a role in prevention of snoring and obstructive sleep apnea by preventing the tongue moving back so as to cause obstruction at the oropharynx resulting in obstructive sleep apnea and snoring. It is provided with supplemental oxygen delivery O₂ system close to the larynx on the back of the tongue from the external source through the oxygen tubing system. The device has 4 holes 141 on the skirt 136 for inserting of the tongs tips or forceps for opening the device and placing it on the tongue.

FIG. 13 is the diagram of the anti-snoring and OSA device insertion forceps (tongs) 142 showing that by approximating the finger holes in the handle, the tip open up (arrows) and thus expands the device. The snoring and obstructive sleep apnea placement forceps have 4 components described as follows:

Blades: The blades are flat which hold the device passing though the holes 141 to grasp the device. Each blade has curves to fit around the tongue out side curvature.

Shanks: The shanks connect the blades to the handles and provide the length of the device to enter the oral cavity. They are parallel, crossing at the lock.

Lock: The lock is the articulation between the shanks with tightening screws to hold the blades containing anti-snoring and obstructive sleep apnea device in position.

Handles: The handles have two finger holes where the operator holds the device opened with thumb and index finger, tighten the lock, holds it on the tip of the tongue, and inserts the device on the tongue gently.

This device placement forceps, the blades tips are passed through the four side holes 141 in front and back of the side of the device sleeve/skirt 136. Then the forceps tips are expanded to open the device to be positioned on the tongue from tip, side and all the way to the back of the tongue as visualized in the FIGS. 11 and 12 with ease. After each use, wash the device placement forceps with warm water and soap or place in platter and immerse in oral antiseptic mouth wash solution. Wash it before using in tepid water.

Use of the device with hard palate and tongue suction cup application with incisor teeth holding attachment without the use of the balloon is all that is needed to provide the anti-snoring and anti-obstructive sleep apnea effect in mild cases of snoring and occasional obstructive sleep apnea sufferers. The anti-snoring and obstructive sleep apnea device embodiments as detailed in the FIGS. 4 to 12 and 14; prevents the development of these conditions during sleep.

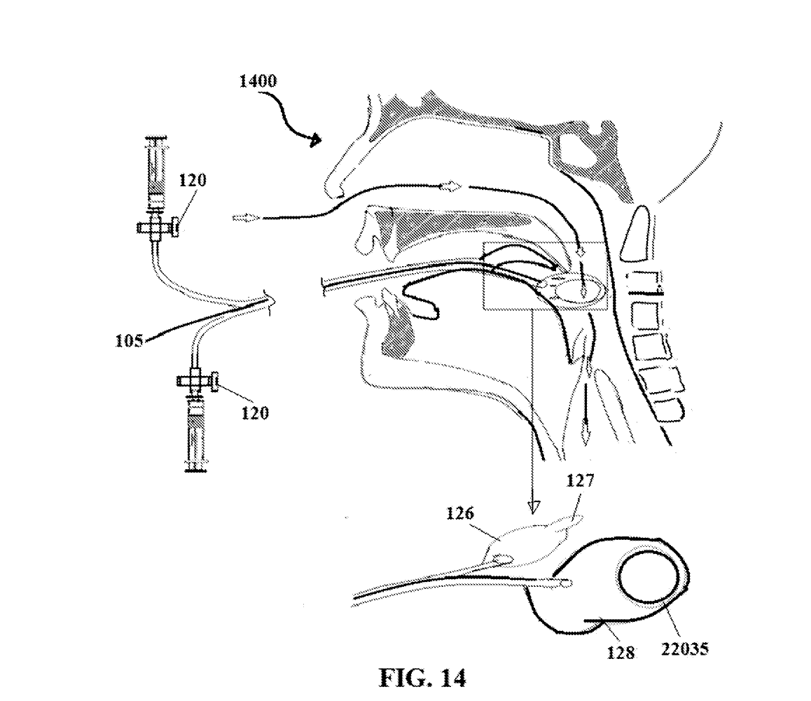

FIG. 14 is the views of the diagram 1400 of the anti-snoring balloon with a ring like expansion 22035 which allows the air to flow from the nose to the larynx without pharyngeal obstruction. The balloon ring 22035 acts as a inlet for the air to pass without obstruction and prevents the tongue falling back on the pharyngeal wall as shown in the FIGS. 2 and 3 during sleep so as to cause obstruction to air flow resulting in snoring and OSA. The palate balloon 126 lifts the soft palate with 127 uvula elevator and separates it from the tongue. It is provided with inflation syringes with three way stop cock 120 and has a string 105 prevents the device being swallowed or aspirated while asleep.

The device is made from vinyl, PVC, plastic, household rubber, silicone, and other synthetic or natural nontoxic, non-allergic composite material. The dorsal surface of the device is made of slightly hardened plastic material or thin metal plate so as to hold the suction cup on the surface and below the surface.

The body of the apparatuses can be made as one piece of plastic material, malleable metal, or silicone or combination thereof. The anti-snoring and obstructive sleep apnea device may be made of thermoplastic or elastomeric resin with metal component such as stainless, aluminum, and copper rods added to strengthen the composite materials. The device can be molded and fashion in such way so that it can be adjusted according to anatomy of users mouth and tongue. The material used to manufacture this device should be non-toxic, non-reacting, and non-allergic.

Preparation of the Patient and Employing the Anti-Snoring and Anti-Obstructive Sleep Apnea Device

Examine the patient thoroughly specially oropharyngeal area, nose, and throat for any medical condition that can predispose snoring with or without obstructive sleep apnea. If there is a contributing factor found, and correctable, the patient should advised to correct it besides using the devices for treatment of snoring and obstructive sleep apnea. If the patient has undergone sleep studies to diagnose sleep apnea, the report need to be obtained to gauge the severity of the OSA. Patients with history of sleep apnea, the sleeping studies are in order.

The patients should be advised to stop smoking, avoid the use of narcotics, sleeping medications, hypnotics, sedative, seep causing antihistamines, and alcohol before going to bed. If the patient is overweight and obese, they should be advised to lose weight and put on weight reduction program.

Advice to avoid food that cause regurgitation (hot foods with spices), which can add to the pathophysiology of OSA, eat moderated supper about 3-4 hours before going to bed. Avoid watching the TV and sleep in a quiet room without any external disturbances that can affect your sleep. The following advice is given before use of anti-snoring and obstructive sleep apnea devices such as 1. Brush the teeth, 2. Use floss to clear the gums. 3. Use tongue scraper to clean the tongue of any coating to prevent bad odor. 4. Use a mild mouth antiseptic wash.

Soak the device in an antiseptic and coat it with lubricant if needed, (oily coating which is not toxic such as menthol olive oil). If the device is not tolerable, due to sensitivity, patient may have to use local anesthetic lozenges, which are available over the counter. If still difficult to use the device due to sensitivity of the oropharyngeal passages, get local anesthetic jelly or spray (Citanest spray) through your physician. Wait till the local anesthetic takes effect and then position the device in the mouth.

Use a well-lighted mirror to position the device with insertion forceps, so as to position the soft palate elevator shelf or balloon is located appropriately at correct position in contact with the oral undersurface of the soft palate, uvula and back of the dorsal surface of the tongue. The device position forceps will also facilitate how deep you need to pass device 101 so that it is placed on the tongue all way to the back the tongue. Make sure the front end is tied to a string provided so that won't be accidentally swallowed or aspirated while sleeping. Use only topical numbing medications when needed. Keep the device clear immersed in antiseptic mouthwash and before use wash in clean warm running water. If oxygen supplementation is needed, use an oxygen concentrator, and keep the flow to the minimum required levels so that it does not disturb sleep with production of sound as it passes through the narrow delivery tubing.

Insert the device, so as to prevent snoring, and one need to insert deep on the surface of the tongue to prevent snoring and OSA. For snoring with obstructive sleep apnea, the tongue shelf restrainer or balloon needs to be carefully positioned properly to prevent the tendency of the flaccid palate and tongue falling or moving posteriorly and inferiorly so as to cause obstruction to the upper respiratory passages which causes snoring and obstructive sleep apnea. Only way to prevent tongue falling back from the floor of the mouth is to prevent obstructive sleep apnea is by mechanical obstruction to prevent the flaccid tongue move backwards during sleep provided by our inventive device with tongue balloon bulge as shown in the FIGS. 11,12 and 14.

If there is no OSA with snoring, use the device with palate shelf as shown in FIG. 6 or balloon as shown in FIG. 11 to prevent movement and vibration of the soft palate. The soft palate becomes flaccid and moves downwards coming in contact with the back of the tongue and oropharynx as one sleeps to produce snoring which is prevented by this device.

The use of the device with balloon is effective for treatment of snoring and obstructive sleep apnea. Blow the balloon slowly after placing the device on the tongue so as to fit the balloon between the soft palate and back of the tongue as close-fitting as one can get to be effective. It there is too much pressure on these structures, deflate the balloon till one feels comfortable. Once the balloon is place and expanded, press the tongue against the hard palate so to activate the vacuum in the suction cups. This will prevents the device moving backwards, and also acts to prevent the tongue move towards the oropharynx due to tongue muscle relaxation (flaccidity) during sleeping. In the morning, when awake, remove the device, wash in running hot water, immerse in antiseptic solution and dry it suspended on the forceps provided.

### ADVANTAGES OF THE CURRENT INVENTION

Advantage of the present invention is that it is available for treating snoring and OSA conditions having an external and internal oral device to reduce or eliminate snoring with or without obstructive sleep apnea during sleep.

An added benefit of the present invention is that it provides for an anti-snoring and anti-OSA device that is easily self-adjustable and do away with the need for professional and laboratory assistance or scientific medical fabrication.

Another benefit of using the described invention is that it has provision for supplemental oxygen for those who have severe pulmonary diseases, needing high concentration of supplemental oxygen to prevent any adverse health effects due to OSA hypoxia during sleep.

Yet another advantage of the present invention is that it provides for an anti-snoring and OSA combined device that is fabricated from a thermoplastic composite material (elastomeric resin and other synthetic material) with or without metal component, which can be easily shaped to fit the anatomy of the oral cavity, tongue, and the soft palate.

An extra benefit of the present invention is that it provides for an anti-snoring and OSA combined device which is moldable after immersion in hot water so that it can be adapted by the user to have a comfortable and individualized fit.

An additional benefit of the present invention is that it provides for an anti-snoring and OSA device that has an intra-oral dental overlay structure—Incisors teeth receptacles or pockets which can be used displaces the lower jaw to the comfortable level at the same time supports the tongue against the user's palate to keep the palate from reverberating during mouth breathing to prevent snoring and OSA by the user.

A further plus of the present invention is that it provides for an anti-snoring and OSA device which can be easily used, stored, cleaned, and mass produced economically to make it affordable for millions who suffer from snoring and obstructive sleep apnea.

Numerous modifications; alternative arrangements of steps explained and examples given herein may be devised by those skilled in the art without departing from the spirit and the scope of the present invention. The appended claims are intended to cover such modifications and arrangements. Thus, the present invention has been described above with particularity and detail in connection. This is presently deemed to be the most practical and preferred embodiments of the invention.

The invention will be apparent to those of ordinary skill in the art that numerous modifications, including, but not limited to, variations in size, materials, shape, form function, manner of procedure, assembly, and the use may be made. The preferred embodiment of the present invention has been described. The invention should be understood that various changes, adaptations, and modifications might be made. It should be understood, therefore, that the invention is not limited to details of the illustrated invention. This method can be used to treat snoring with or without obstructive sleep apnea and prevent the health hazards associated with the conditions.

Although the instant invention has been described in relation to particular embodiments thereof, many other variations and modifications and other uses will become apparent to those skilled in the art.

What is claimed is:

1. An anti-snoring and anti-obstructive sleep apnea device comprising:
an oral device portion adapted to removably cover substantially all of a top portion of a tongue;
said oral device portion covering a tip and side portions of said tongue wherein said oral device portion is securely held in place;
a plate portion centrally disposed on said oral device portion;
a suction cup disposed on an upper and lower surface of said plate portion wherein said suction cup removably holds said oral device portion to a dorsal surface of said tongue;
an inflatable balloon disposed on a posterior portion of said plastic plate;
said inflatable balloon being adapted to contact a portion of a soft palate, an uvula and a back portion of said tongue wherein said portion of soft palate, uvula, and back portion of said tongue is prevented from moving backwards whereby snoring and obstructive sleep apnea are prevented during use; and
said inflatable balloon having an airway opening centrally disposed therein wherein air is allowed to move through said airway opening from a nose to a larynx.

2. The anti-snoring and anti-obstructive sleep apnea device according to claim 1 further comprising:
a hood portion adapted to fit across said tip of said tongue wherein said oral device portion is prevented from moving back on said tongue; and;
said hood portion being disposed on a forward portion of said oral device portion.

3. The anti-snoring and anti-obstructive sleep apnea device according to claim 1 further comprising at least one additional suction cup disposed on said plate portion wherein said oral device portion is secured against said tongue by making contact against a hard palate portion and dorsum of said tongue.

4. The anti-snoring and anti-obstructive sleep apnea device according to claim 1 wherein said suction cup is adapted to hold a dental adhesive.

5. The anti-snoring and anti-obstructive sleep apnea device according to claim 1 further comprising a safety portion attached to said oral device portion wherein said anti-snoring and anti-obstructive sleep apnea device is prevented from being accidentally swallowed during sleep.

6. The anti-snoring and anti-obstructive sleep apnea device according to claim 1 further comprising a three way stopcock and syringe adapted to allow inflation from outside the mouth.

7. An anti-snoring and anti-obstructive sleep apnea device comprising:
an oral device portion adapted to removably cover substantially all of a top portion of a tongue;
said oral device portion covering a tip and side portions of said tongue wherein said oral device portion is securely held in place;
a plate portion centrally disposed on said oral device portion;
a suction cup disposed on an upper and lower surface of said plate portion wherein said suction cup removably holds said oral device portion to a dorsal surface of said tongue;
a moldable soft palate shelf disposed on a posterior portion of said plastic plate; and
said moldable soft palate shelf being adapted to prevent said tongue from moving backwards whereby snoring and obstructive sleep apnea are prevented during use.

8. The anti-snoring and anti-obstructive sleep apnea device according to claim 1 further comprising:
a plurality of oxygen delivery exits disposed on a posterior portion of said oral device portion; and
an oxygen delivery tubing portion in communication with said oxygen delivery exits and an oxygen source wherein hypoxia is prevented especially in pulmonary compromised patients.

9. The anti-snoring and anti-obstructive sleep apnea device according to claim 8 wherein said oxygen source is an external supplemental oxygen supply.

10. The anti-snoring and anti-obstructive sleep apnea device according to claim 9 further comprising;
a tooth socket;
said tooth socket having a “V”-shaped configuration, whereby an incisor tooth fits within said tooth socket wherein said tongue is prevented from sliding back.

11. The anti-snoring and anti-obstructive sleep apnea device according to claim 11 further comprising a metallic strip embedded therein to strengthen said oral device portion.

12. The anti-snoring and anti-obstructive sleep apnea device according to claim 7 wherein said oral device portion is made of moldable thermoplastic.

13. The anti-snoring and anti-obstructive sleep apnea device according to claim 7 wherein said oral device portion is made of elastomeric resin.

14. The anti-snoring and anti-obstructive sleep apnea device according to claim 7 wherein said oral device portion is made of plastic.

15. The anti-snoring and anti-obstructive sleep apnea device according to claim 7 wherein said oral device portion is made of a composite material.

16. The anti-snoring and anti-obstructive sleep apnea device according to claim 7 wherein said oral device portion is made of a metallic material.

17. The anti-snoring and anti-obstructive sleep apnea device according to claim 1 wherein said oral device portion is selected to match a particular oropharyngeal anatomy and pathophysiology.

18. The anti-snoring and anti-obstructive sleep apnea device according to claim 1 wherein said oral device portion is adapted to allow a user to easily remove and reinsert in a mouth therein whereby talking, yawning, coughing and clearing the throat is facilitated.
